(19) United States
(12) Patent Application Publication
(10) Pub. No.: US 2019/0104930 A1
(43) Pub. Date: Apr. 11, 2019

(54) OPTICAL FIBER SCANNER, ILLUMINATION APPARATUS, AND OBSERVATION APPARATUS

(71) Applicant: OLYMPUS CORPORATION, Tokyo (JP)

(72) Inventors: Takashi YASUMI, Tokyo (JP); Yasuaki KASAI, Saitama (JP); Hirokazu YOKOTA, Tokyo (JP); Hiroshi TSURUTA, Kanagawa (JP)

(73) Assignee: OLYMPUS CORPORATION, Tokyo (JP)

(21) Appl. No.: 16/206,031

(22) Filed: Nov. 30, 2018

Related U.S. Application Data
(63) Continuation of application No. PCT/JP2017/021670, filed on Jun. 12, 2017.

(30) Foreign Application Priority Data
Jun. 14, 2016 (JP) PCT/JP2016/067654

(51) Int. Cl. A61B 1/00; A61B 1/07; G02B 23/24; G02B 26/10

(52) U.S. Cl. CPC A61B 1/00172; A61B 1/07; G02B 26/103; G02B 23/2469; G02B 23/2476; G02B 23/2423

(57) ABSTRACT

Provided is an optical fiber scanner including: an optical fiber that guides light from a side of a proximal-end portion to a side of a distal-end portion along a longitudinal axis, the optical fiber emitting the light from the distal-end portion; a piezoelectric element that is secured to an outer circumferential surface of the optical fiber, the piezoelectric element, as a result of an alternating voltage being applied thereto, generating stretching vibrations in a direction along the longitudinal axis; and a pressing portion that, of an outer surface of the piezoelectric element positioned on the radially outer side of the optical fiber, presses down, radially inward, a portion corresponding to an antinode of the stretching vibrations in a direction along the longitudinal axis of the piezoelectric element.

### CROSS-REFERENCE TO RELATED APPLICATIONS

This is a continuation of International Application PCT/JP2017/021670, with an international filing date of Jun. 12, 2017, which is hereby incorporated by reference herein in its entirety.

This application is based on International Application PCT/JP2016/067654, the contents of which are incorporated herein by reference.

### TECHNICAL FIELD

The present invention relates to an optical fiber scanner, an illumination apparatus, and observation apparatus.

### BACKGROUND ART

In the related art, there is a known optical fiber scanner with which light emitted from a distal end of an optical fiber is scanned by causing the distal end of the optical fiber to vibrate by means of piezoelectric elements (for example, see Patent Literature 1). In the optical fiber scanner described in Patent Literature 1, the piezoelectric elements are secured to an outer circumferential surface of the optical fiber via ferrules. As a result of the vibrations generated by the piezoelectric elements by applying voltages thereto being propagated to the optical fiber via the ferrules, the distal end of the optical fiber is caused to vibrate.

### CITATION LIST

### Patent Literature

PTL 1 Japanese Unexamined Patent Application, Publication No. 2013-244045

### SUMMARY OF INVENTION

A first aspect of the present invention is an optical fiber scanner including: an optical fiber that guides light from a side of a proximal-end portion to a side of a distal-end portion along a longitudinal axis, the optical fiber emitting the light from the distal-end portion; a piezoelectric element that is secured to an outer circumferential surface of the optical fiber, the piezoelectric element, as a result of an alternating voltage being applied thereto, generating stretching vibrations in a direction along the longitudinal axis; and a pressing portion that, of an outer surface of the piezoelectric element positioned on the radially outer side of the optical fiber, presses down, radially inward, a portion corresponding to an antinode of the stretching vibrations in a direction along the longitudinal axis of the piezoelectric element.

A second aspect of the present invention is an illumination apparatus including: an optical fiber scanner according to the above-described first aspect; and a light source portion that is connected to the proximal-end portion of the optical fiber, the light source portion supplying the light to the optical fiber.

A third aspect of the present invention is an observation apparatus including: an illumination apparatus according to the above-described second aspect; a light detecting portion that detects return light returning from an imaging subject, which is generated by the imaging subject being irradiated with the light coming from the illumination apparatus; and a voltage supplying portion that supplies the alternating voltage to the piezoelectric element.

### BRIEF DESCRIPTION OF DRAWINGS

FIG. 1 is an overall configuration diagram showing an observation apparatus provided with an optical fiber scanner and an illumination apparatus according to an embodiment of the present invention.

FIG. 2 is a longitudinal cross-sectional view taken along a longitudinal axis, showing the internal configuration of a distal end of an inserted portion of an endoscope of the observation apparatus in FIG. 1.

FIG. 3A is a side view showing an overall configuration of the optical fiber scanner according to a first embodiment of the present invention.

FIG. 3B is a front view in which the optical fiber scanner in FIG. 3A is viewed from a distal-end side.

FIG. 4 is a side view showing the overall configuration of a modification of the optical fiber scanner in FIG. 3A.

FIG. 5 is a side view showing the overall configuration of another modification of the optical fiber scanner in FIG. 3A.

FIG. 6 is a side view showing the overall configuration of another modification of the optical fiber scanner in FIG. 3A.

FIG. 7A is a side view showing the overall configuration of another modification of the optical fiber scanner in FIG. 3A.

FIG. 7B is a front view in which the optical fiber scanner in FIG. 7A is viewed from a distal-end side.

FIG. 8A is a side view showing the overall configuration of another modification of the optical fiber scanner in FIG. 3A.

FIG. 8B is a front view in which the optical fiber scanner in FIG. 8A is viewed from a distal-end side.

FIG. 8C shows a cross-sectional view (left) and a front view (right) of a pressing portion of the optical fiber scanner in FIG. 8A.

FIG. 9A is a side view showing the overall configuration of another modification of the optical fiber scanner in FIG. 3A.

FIG. 9B is a front view in which the optical fiber scanner in FIG. 9A is viewed from a distal-end side.

FIG. 9C shows a cross-sectional view (left) and a front view (right) of a pressing portion of the optical fiber scanner in FIG. 9A.

FIG. 10A is a side view showing the overall configuration of another modification of the optical fiber scanner in FIG. 3A.

FIG. 10B is a front view in which the optical fiber scanner in FIG. 10A is viewed from a distal-end side.

FIG. 10C shows a cross-sectional view (left) and a front view (right) of a pressing portion of the optical fiber scanner in FIG. 10A.

FIG. 11A is a side view showing the overall configuration of another modification of the optical fiber scanner in FIG. 3A.

FIG. 11B is a front view in which the optical fiber scanner in FIG. 11A is viewed from a distal-end side.

### DESCRIPTION OF EMBODIMENT

An optical fiber scanner 1, an illumination apparatus 10, and an observation apparatus 100 according to an embodiment of the present invention will be described with reference to the drawings.

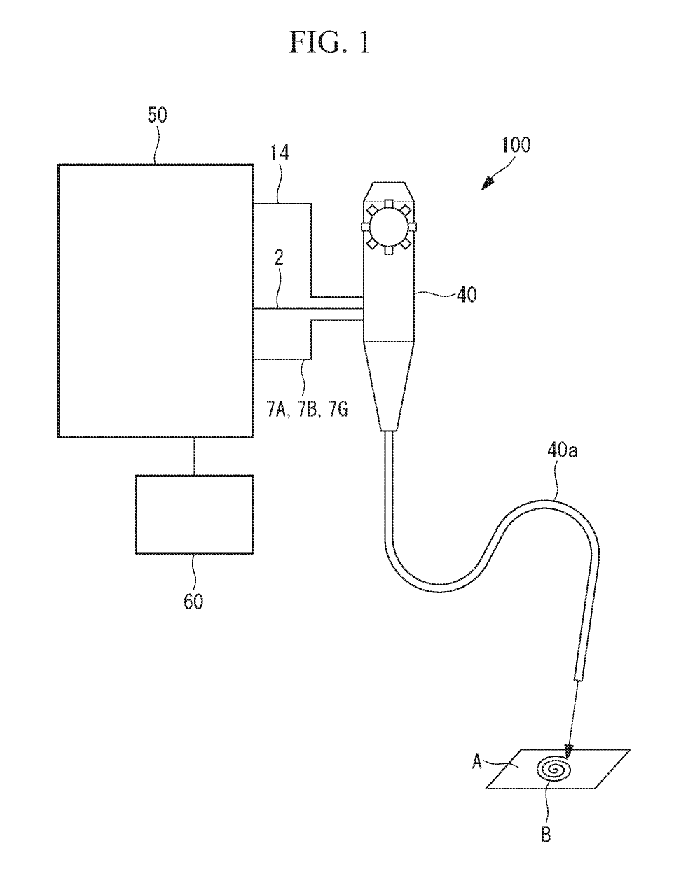

As shown in FIG. 1, the observation apparatus 100 according to this embodiment is provided with an endoscope 40 having a long, thin inserted portion 40a, a control apparatus main unit 50 connected to the endoscope 40, and a display 60 connected to the control apparatus main unit 50. The observation apparatus 100 is a light-scanning endoscope apparatus that two-dimensionally scans illumination light emitted from a distal end of the inserted portion 40a on an imaging subject A along a spiral scanning trajectory B, thereby acquiring an image of the imaging subject A.

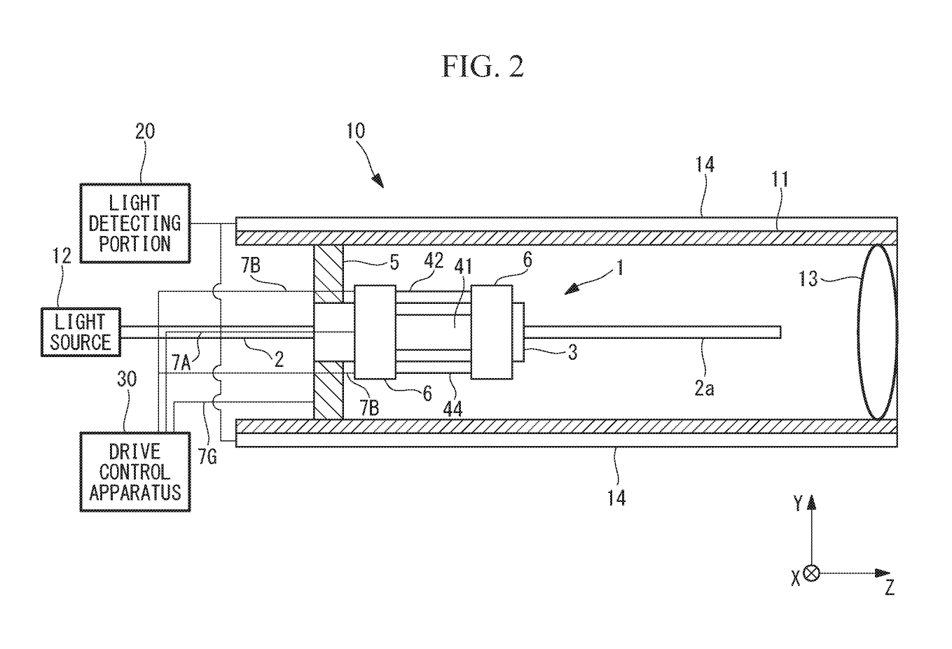

As shown in FIG. 2, the observation apparatus 100 is provided with: the illumination apparatus 10 that radiates the illumination light onto the imaging subject A; a light detecting portion 20 that has a light detector such as a photodiode and that detects return light returning from the imaging subject A as a result of the illumination light being radiated onto the imaging subject A; and a drive control apparatus (voltage supplying portion) 30 that controls driving of the illumination apparatus 10 and the light detecting portion 20. The light detecting portion 20 and the drive control apparatus 30 are provided in the control apparatus main unit 50.

The illumination apparatus 10 is provided with: a long, thin cylindrical frame 11 provided in the inserted portion 40a; a light source (light source portion) 12 that is provided in the control apparatus main unit 50 and that generates the illumination light; the optical fiber scanner 1 that is provided in the frame 11 and that has an illumination optical fiber 2 that guides the illumination light emitted from the light source 12 from a proximal end to a distal end and emits the light from the distal end; a focusing lens 13 that is disposed in the frame 11 so as to be closer to the distal end than the optical fiber 2 is and that focuses the illumination light emitted from the optical fiber 2; and a plurality of detection optical fibers 14 that are provided on an outer circumferential surface of the frame 11 so as to be arrayed in a circumferential direction, and that guide the return light (for example, reflected light of the illumination light or fluorescence) coming from the imaging subject A toward the light detecting portion 20.

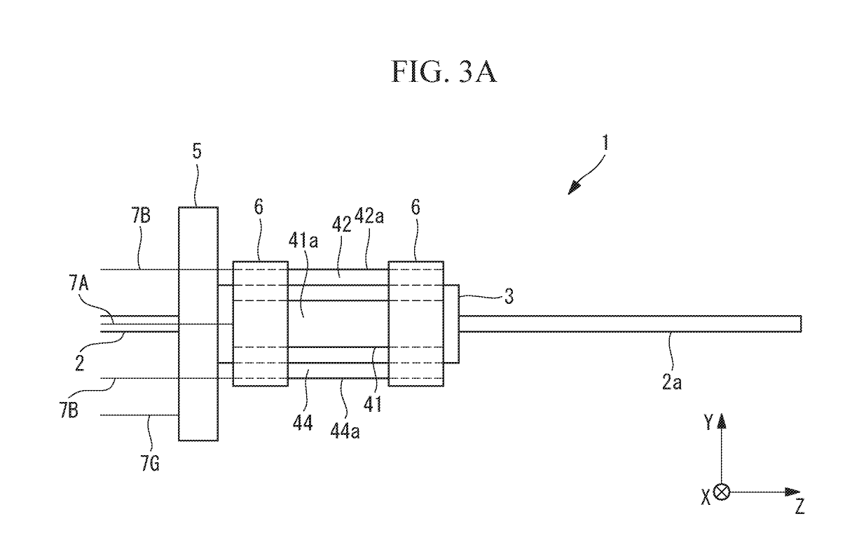

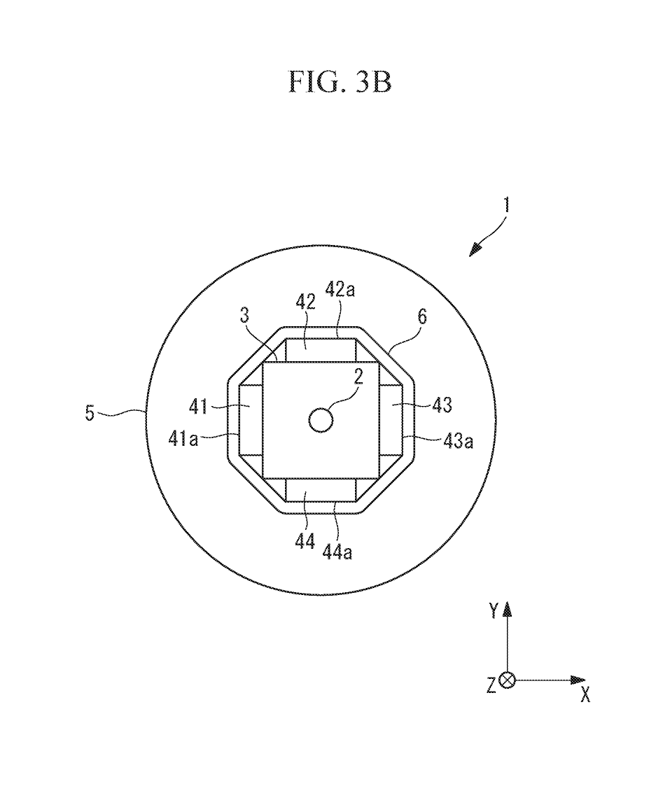

As shown in FIGS. 3A and 3B, the optical fiber scanner 1 is provided with: the optical fiber 2; a cylindrical vibration propagating portion 3 that is secured to an outer circumferential surface of the optical fiber 2; a plurality of piezoelectric elements 41, 42, 43, and 44 that are secured to outer circumferential surfaces of the vibration propagating portion 3; a securing portion 5 that is provided so as to be closer to the proximal end than the piezoelectric elements 41, 42, 43, and 44 are and that secures the optical fiber 2 to the frame 11; and pressing portions 6 that press the piezoelectric elements 41, 42, 43, and 44 against the vibration propagating portion 3.

The optical fiber 2 is a multi-mode fiber or a single-mode fiber, and is formed of a columnar glass material having a longitudinal axis. The optical fiber 2 is disposed in the frame 11 along the longitudinal direction, the distal end of the optical fiber 2 is disposed in the vicinity of a distal-end portion of the interior of the frame 11, and a proximal end of the optical fiber 2 is connected to the light source 12. In the following, the longitudinal direction of the optical fiber 2 is assumed to be a Z-direction, and two radial directions of the optical fiber 2 that are orthogonal to each other are assumed to be an X-direction and a Y-direction.

The vibration propagating portion 3 is formed of a rectangular tubular member having a through-hole that passes therethrough along a center axis thereof, and the optical fiber 2 is inserted into the through-hole. The vibration propagating portion 3 is provided at a position that is closer to a proximal-end portion of the optical fiber 2 than to the distal-end portion of the optical fiber 2, and an inner circumferential surface of the through-hole is secured to an outer circumferential surface of the optical fiber 2 by using an adhesive. In the following, the distal-end portion of the optical fiber 2 that protrudes from the distal-end surface of the vibration propagating portion 3 toward the distal end is referred to as a protrusion 2a. The vibration propagating portion 3 is formed of a metal possessing elasticity (for example, nickel, stainless steel, iron, an aluminum alloy, or titanium).

The piezoelectric elements 41, 42, 43, and 44 are rectangular flat plates that are formed of a piezoelectric ceramic material such as lead zirconate titanate (PZT). In the piezoelectric elements 41, 42, 43, and 44, electrotreatment is applied to two end surfaces thereof that face each other in the thickness direction so as to be polarized in the thickness direction. The two A-phase piezoelectric elements 41 and 43 are individually secured, by means of an adhesive, to two side surfaces of the vibration propagating portion 3 that face each other in the X-direction so that the polarization direction becomes parallel to the X-direction. The two B-phase piezoelectric elements 42 and 44 are individually secured, by means of the adhesive, to two side surfaces of the vibration propagating portion 3 that face each other in the Y-direction so that the polarization becomes parallel to the Y-direction.

The securing portion 5 is a cylindrical member having a greater external size than that of the vibration propagating portion 3, and the proximal-end portion of the vibration propagating portion 3 is inserted into the securing portion 5. An inner circumferential surface of the securing portion 5 is secured to the proximal-end portion of the vibration propagating portion 3, and an outer circumferential surface of the securing portion 5 is secured to an inner wall of the frame 11. By doing so, the vibration propagating portion 3 and the protrusion 2a of the optical fiber 2 are supported by the securing portion 5 in a cantilever-like manner with the distal ends thereof being free ends. The securing portion 5 is electrically connected to the vibration propagating portion 3 via the piezoelectric elements 41, 42, 43, and 44, and serves as a common ground (GND) when alternating voltages are applied to the piezoelectric elements 41, 42, 43, and 44.

A-phase lead lines 7A are individually connected to the two A-phase piezoelectric elements 41 and 43 by using a conductive adhesive. B-phase lead lines 7B are individually connected to the two B-phase piezoelectric elements 42 and 44 by using of the conductive adhesive. A GND lead line 7G is connected to the securing portion 5 by using the conductive adhesive. The lead lines 7A, 7B, and 7G are individually connected to the drive control apparatus 30. In FIG. 3B, illustrations of the lead lines 7A, 7B, and 7G are omitted.

The pressing portions 6 are formed of annular members that generate a contractile force in a circumferential direction, for example, heat contraction tubes, rubber rings, or flat rubber members. The pressing portions 6 wrap around periphery of the vibration propagating portion 3 and the piezoelectric elements 41, 42, 43, and 44. Of the two end surfaces of the individual piezoelectric elements 41, 42, 43, and 44, end surfaces (outer surfaces) 41a, 42a, 43a, and 44a that are positioned at the outer side in the X-direction or the Y-direction come into contact with the pressing portions 6 that are contracted by the contractile force, and the pressing portions 6 press down the individual outer surfaces 41a, 42a, 43a, and 44a radially inward with respect to the optical fiber 2. In the individual outer surfaces 41a, 42a, 43a, and 44a, the pressing portions 6 are provided at two locations, namely, at a distal-end portion and a proximal-end portion in the longitudinal direction, and press down only the distal-end portion and the proximal-end portion.

The lead lines 7A and 7B are connected to proximal-end portions of the outer surfaces 41a, 42a, 43a, and 44a. The pressing portions 6 are provided so as to sandwich the lead lines 7A and 7B between the outer surfaces 41a, 42a, 43a, and 44a.

The drive control apparatus 30 applies A-phase alternating voltages to the piezoelectric elements 41 and 43 via the A-phase lead lines 7A, and applies B-phase alternating voltages to the piezoelectric elements 42 and 44 via the B-phase lead lines 7B.

When the alternating voltages are applied to the A-phase piezoelectric elements 41 and 43, the piezoelectric elements 41 and 43 generate stretching vibrations in the Z-direction, bending vibrations in the X-direction are excited in the vibration propagating portion 3, and thus, the bending vibrations in the vibration propagating portion 3 are propagated to the optical fiber 2. By doing so, the illumination light emitted from the distal end of the optical fiber 2 is scanned in the X-direction. When the alternating voltages are applied to the B-phase piezoelectric elements 42 and 44, the piezoelectric elements 42 and 44 generate stretching vibrations in the Z-direction, bending vibrations in the Y-direction are excited in the vibration propagating portion 3, and thus, the bending vibrations in the vibration propagating portion 3 are propagated to the optical fiber 2. By doing so, the illumination light emitted from the distal end of the optical fiber 2 is scanned in the Y-direction. Therefore, by controlling the amplitudes and phases of the alternating voltages to be applied to the piezoelectric elements 41, 42, 43, and 44, it is possible to control the scanning trajectory B of the illumination light.

Next, the operations of the thus-configured optical fiber scanner 1, illumination apparatus 10, and observation apparatus 100 will be described.

In order to observe the imaging subject A by using the observation apparatus 100 according to this embodiment, the drive control apparatus 30 is activated, the illumination light is supplied to the optical fiber 2 from the light source 12, and the alternating voltages are applied to the piezoelectric elements 41, 42, 43, and 44 via the lead lines 7A and 7B.

The piezoelectric elements 41, 42, 43, and 44 to which the alternating voltages have been applied individually generate the stretching vibrations in the Z-direction, and excite the bending vibrations in the vibration propagating portion 3 and the protrusion 2a of the optical fiber 2. By doing so, the distal end of the optical fiber 2 vibrates in the radial direction, thus scanning the illumination light emitted from the distal end of the optical fiber 2 on the imaging subject A. The return light coming from the imaging subject A is received by the plurality of optical fibers 14, and the intensity thereof is detected by the light detecting portion 20. The drive control apparatus 30 generates an image of the imaging subject A by associating the detected return light intensities with scanning positions of the illumination light. The generated image is displayed on the display 60.

In this case, unevenness occurs in the joining layers formed of the adhesive between the piezoelectric elements 41, 42, 43, and 44 and the vibration propagating portion 3 due to various factors in the manufacturing process. For example, pores unevenly occur in the joining layers due to the air remaining in the adhesive before being cured. As a result of the piezoelectric elements 41, 42, 43, and 44 and the vibration propagating portion 3 being unevenly bonded to each other, the efficiency at which the vibrations are propagated to the vibration propagating portion 3 and the optical fiber 2 from the piezoelectric elements 41, 42, 43, and 44 could decrease.

With this embodiment, the piezoelectric elements 41, 42, 43, and 44 evenly come into contact with the vibration propagating portion 3 via the joining layers as a result of the piezoelectric elements 41, 42, 43, and 44 being pressed against the vibration propagating portion 3 by the pressing portions 6, and thus, the efficiency at which vibrations are propagated to the vibration propagating portion 3 and the optical fiber 2 from the piezoelectric elements 41, 42, 43, and 44 is enhanced. Accordingly, there is an advantage in that it is possible to increase the vibration amplitude of the distal end of the optical fiber 2 with respect to the magnitude of the alternating voltage. In addition, there is an advantage in that it is possible to more stably secure the piezoelectric elements 41, 42, 43, and 44 to the vibration propagating portion 3 by means of the pressing portions 6.

In particular, by pressing down the outer surfaces 41a, 42a, 43a, and 44a of the piezoelectric elements 41, 42, 43, and 44 by means of the pressing portions 6 at the distal-end portion and the proximal-end portion which correspond to antinodes of the stretching vibrations, vibrations at maximum displacement positions of the piezoelectric elements 41, 42, 43, and 44 are more efficiently transmitted to the vibration propagating portion 3 and the optical fiber 2, as compared with the case in which other portions of the piezoelectric elements 41, 42, 43, and 44 are pressed down by the pressing portions 6. Accordingly, there is an advantage in that it is possible to more efficiently increase the vibration amplitude of the distal end of the optical fiber 2.

In addition, by pressing down the positions at which the lead lines 7A and 7B are connected to the outer surfaces 41a, 42a, 43a, and 44a with pressing portions 6, there is an advantage in that it is possible to more stably maintain the connections between the lead lines 7A and 7B and the outer surfaces 41a, 42a, 43a, and 44a.

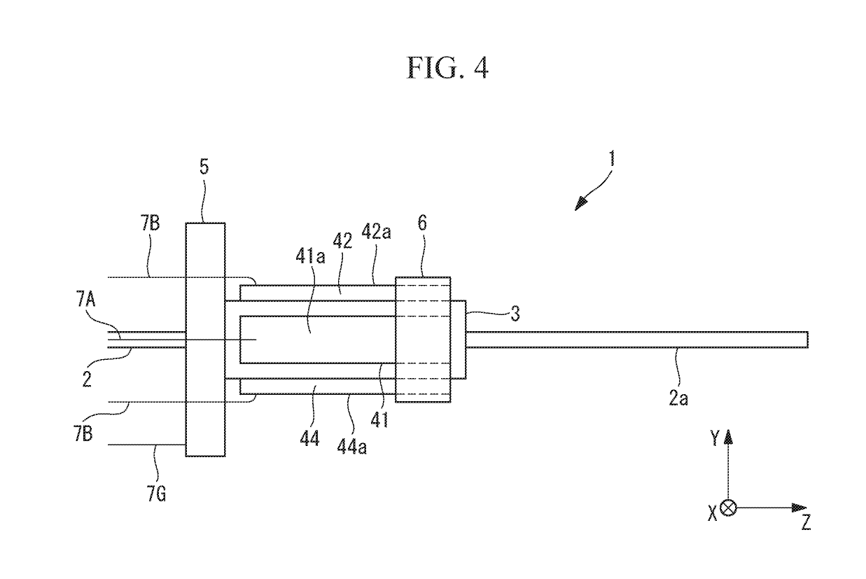

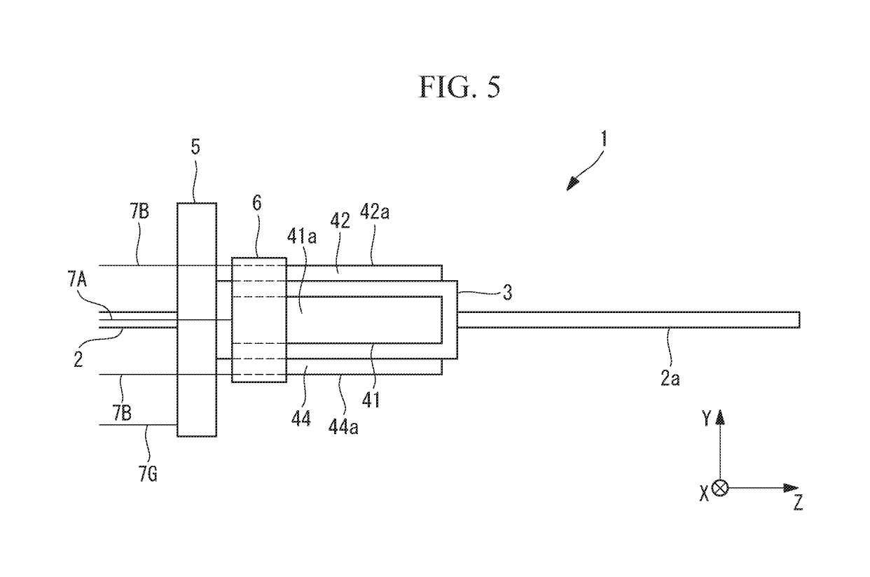

In this embodiment, although the distal-end portions and the proximal-end portions of the outer surfaces 41a, 42a, 43a, and 44a of the piezoelectric elements 41, 42, 43, and 44 are both pressed down with the pressing portions 6, alternatively, the pressing portion 6 may be provided only at the distal-end portions, as shown in FIG. 4, or the pressing portion 6 may be provided only at the proximal-end portions, as shown in FIG. 5. When the optical fiber scanner 100 is viewed from the front, the configuration thereof is the same as the configuration of the optical fiber scanner 1 shown in FIG. 3B. By doing so also, it is possible to efficiently transmit the vibrations at the maximum displacement positions of the piezoelectric elements 41, 42, 43, and 44 to the vibration propagating portion 3 and the optical fiber 2.

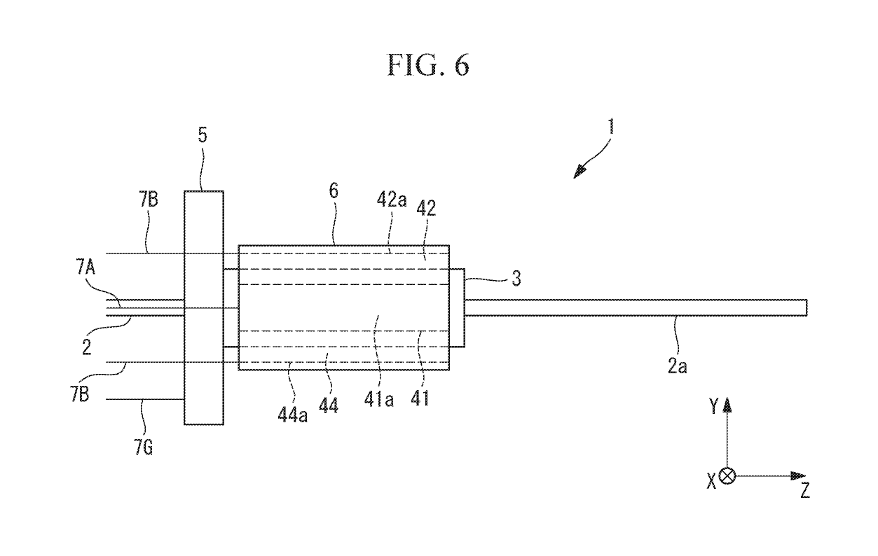

In this embodiment, although the pressing portions 6 are provided only at the distal-end portions and the proximal-end portions of the outer surfaces 41a, 42a, 43a, and 44a, alternatively, as shown in FIG. 6, a pressing portion 6 may be provided over the entire lengths of the outer surfaces 41a, 42a, 43a, and 44a in the longitudinal direction so as to press down the outer surfaces 41a, 42a, 43a, and 44a over the entire lengths thereof in the longitudinal direction.

By doing so also, by pressing down, by means of the pressing portion 6, the outer surfaces 41a, 42a, 43a, and 44a of the piezoelectric elements 41, 42, 43, and 44 at the proximal-end portions thereof, which correspond to antinodes of the stretching vibrations, it is possible to efficiently transmit the vibrations at the maximum displacement positions of the piezoelectric elements 41, 42, 43, and 44 to the vibration propagating portion 3 and the optical fiber 2.

Furthermore, in the case in which the pressing portion 6 is formed of an electrical insulator, there is an advantage in that it is possible to electrically insulate the periphery of the piezoelectric elements 41, 42, 43, and 44 by means of the pressing portion 6 that covers the enter outer surfaces 41a, 42a, 43a, and 44a.

In this embodiment, although the pressing portion 6 is formed of an annular member that generates a contractile force, such as a heat contraction tube or an annular rubber member, any member may be employed as the pressing portion 6 so long as said member is capable of pressing down the outer surfaces 41a, 42a, 43a, and 44a radially inward.

For example, the pressing portion 6 may be a string-like member that ties the four piezoelectric elements 41, 42, 43, and 44 down to the vibration propagating portion 3.

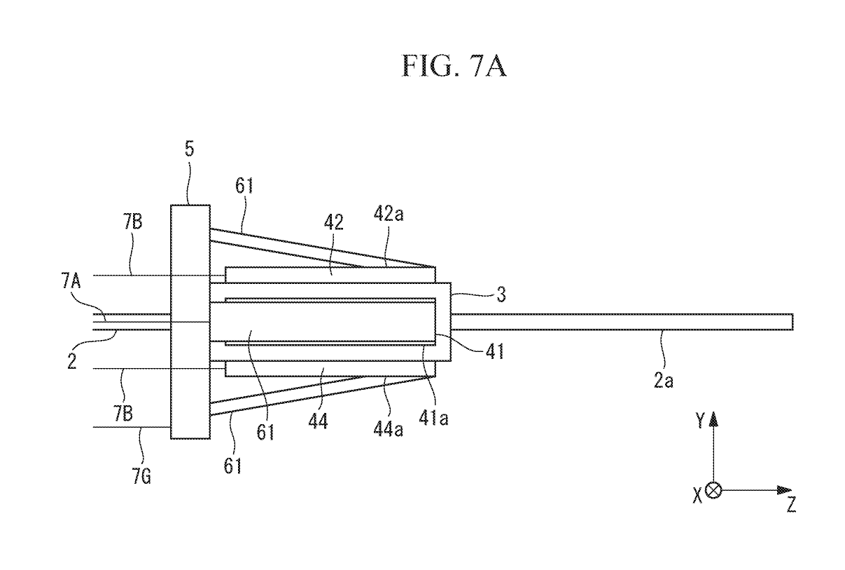

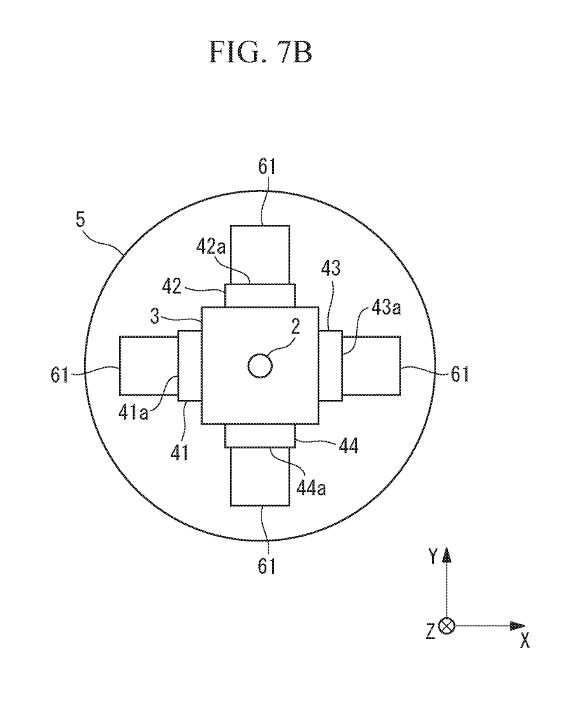

Alternatively, as shown in FIGS. 7A and 7B, pressing portion may be constituted of plate springs 61 that are provided so as to correspond to the individual piezoelectric elements 41, 42, 43, and 44, and that press down the corresponding piezoelectric elements 41, 42, 43, and 44 radially inward.

One end of the each of the plate springs 61 is secured to the securing portion 5. At the other end of each plate spring 61, a pressing surface that comes into contact with the outer surface 41a, 42a, 43a, or 44a and that presses down the outer surface 41a, 42a, 43a, or 44a is formed. By doing so also, by pressing the piezoelectric elements 41, 42, 43, and 44 against the vibration propagating portion 3 by means of the plate springs 61, it is possible to efficiently cause the distal end of the optical fiber 2 to vibrate.

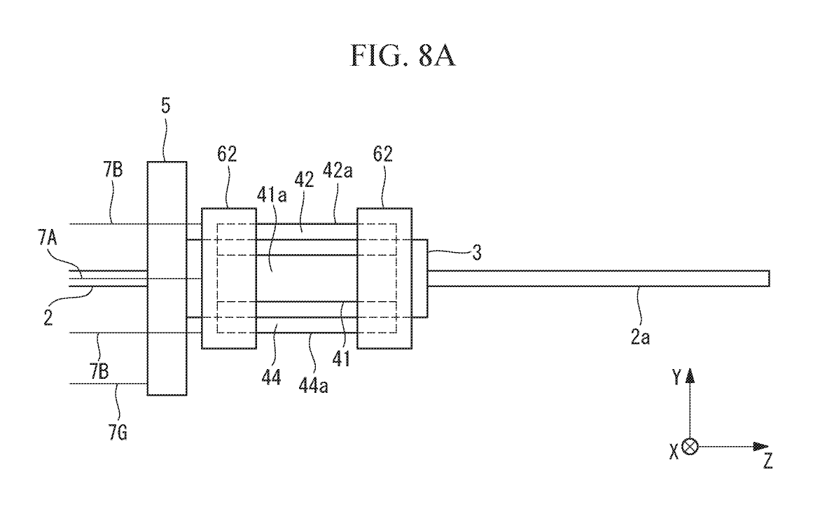

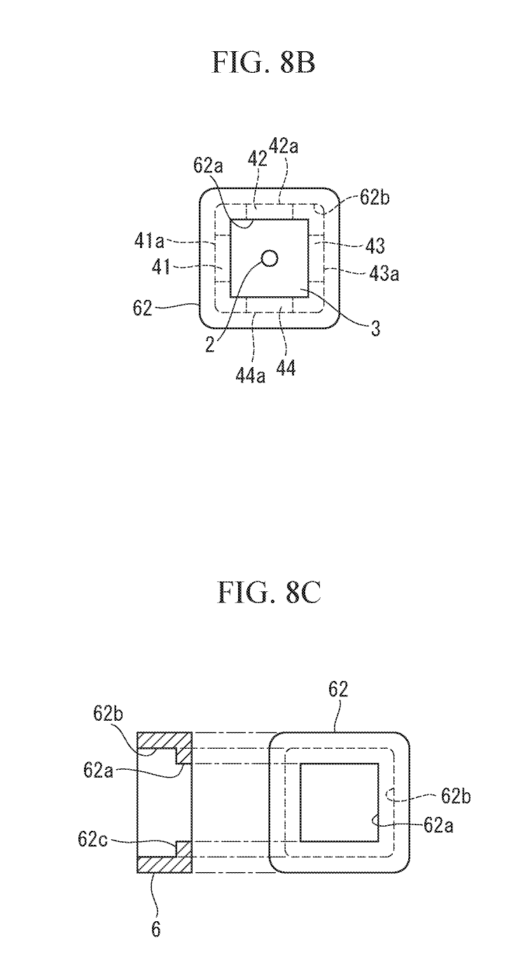

In this embodiment, although the pressing portion 6 is formed of an annular member, and presses down only the outer surfaces 41a, 42a, 43a, and 44a of the piezoelectric elements 41, 42, 43, and 44 radially inward, alternatively, as shown in FIGS. 8A to 8C, pressing portions 62 may press down the distal-end surfaces and the proximal-end surfaces of the piezoelectric elements 41, 42, 43, and 44 in a direction along the longitudinal axis of the optical fiber 2.

Specifically, the pressing portions 62 have through-holes 62a through which the vibration propagating portion 3 passes and depressions 62b that have a greater diameter than those of the through-holes 62a and that receive end portions of the piezoelectric elements 41, 42, 43, and 44. Therefore, inner surfaces of the pressing portions 62 have a step shape formed of two steps constituted of inner surfaces of the through-holes 62a and inner surfaces of the depressions 62b, and annular abutting surfaces 62c are formed between the through-holes 62a and the depressions 62b. The distal-end portions and the proximal-end portions of the piezoelectric elements 41, 42, 43, and 44 individually abut against the abutting surfaces 62c in a direction along the longitudinal axis. The pressing portions 62 are secured to the vibration propagating portion 3 at the inner circumferential surfaces of the through-holes 62a, and are secured to the piezoelectric elements 41, 42, 43, and 44 at the inner circumferential surfaces of the depressions 62b and the abutting surfaces 62c.

In this way, by employing the pressing portions 62 provided with the steps, the piezoelectric elements 41, 42, 43, and 44 and the pressing portions 62 are positioned with respect to each other in the direction along the longitudinal direction so that the pressing portions 62 are disposed at appropriate positions with respect to the maximum displacement positions of the piezoelectric elements 41, 42, 43, and 44. By doing so, it is possible to enhance the assembly precision between the piezoelectric elements 41, 42, 43, and 44 and the pressing portions 62, and thus, it is possible to more efficiently transmit the vibrations at the maximum displacement positions of the piezoelectric elements 41, 42, 43, and 44 to the vibration propagating portion 3 and the optical fiber 2.

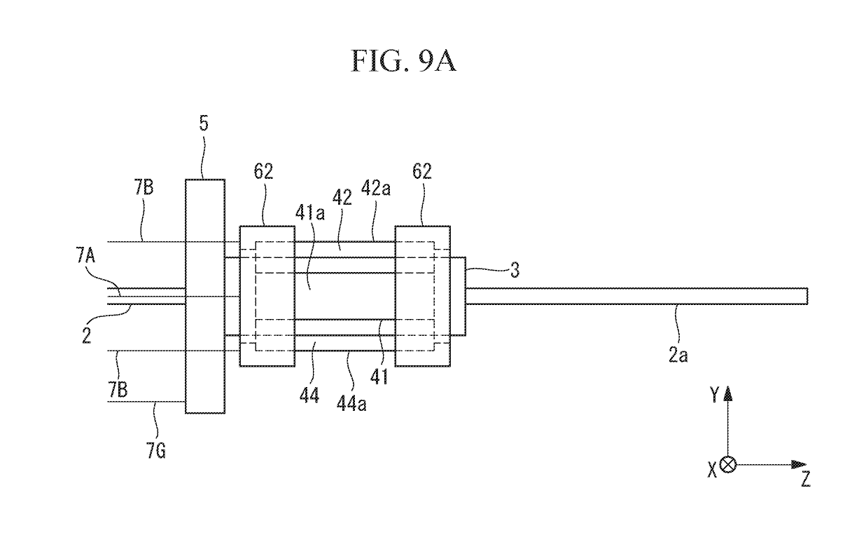

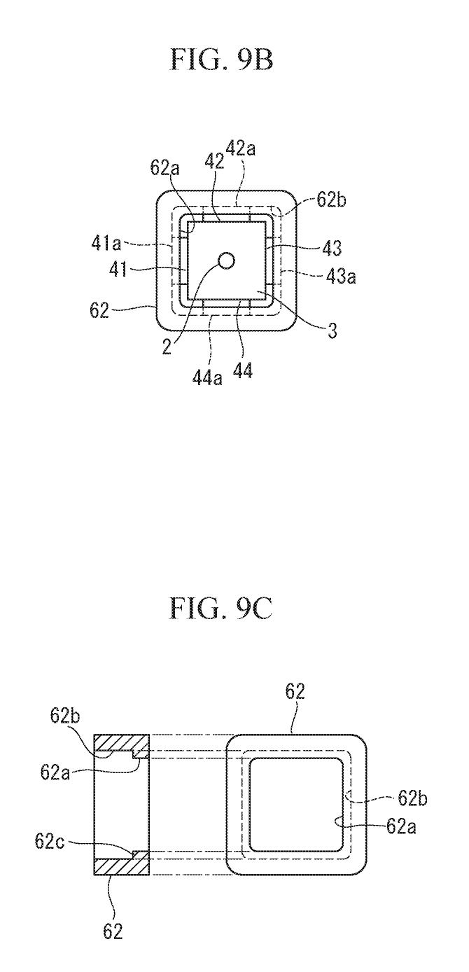

As shown in FIGS. 9A to 9C, the inner circumferential surfaces of the through-holes 62a may be separated from the vibration propagating portion 3, and the pressing portions 62 may be secured only to the piezoelectric elements 41, 42, 43, and 44.

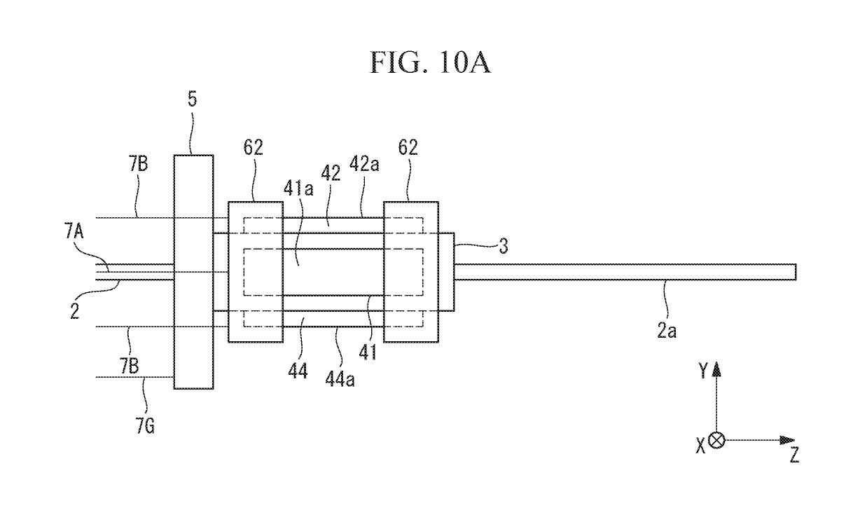

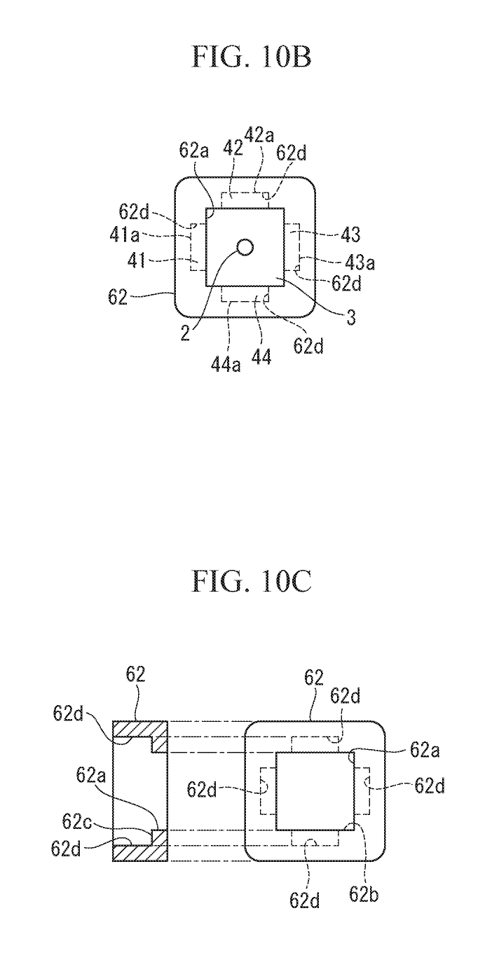

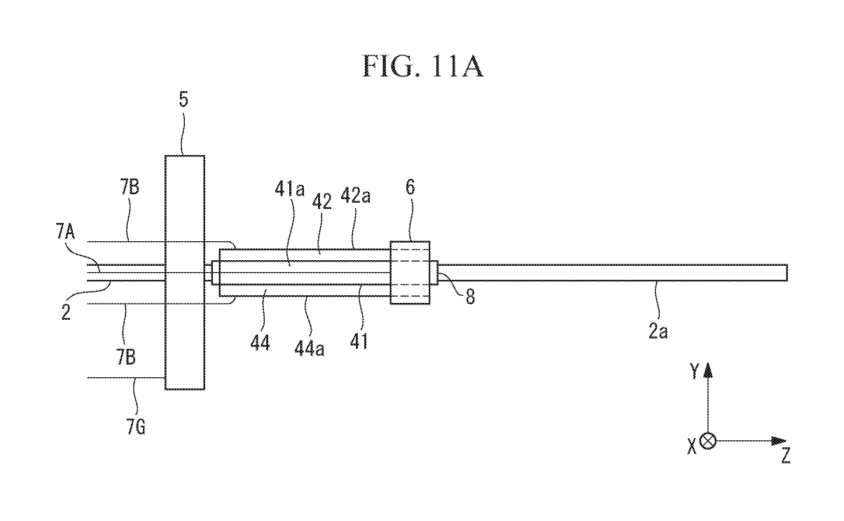

In addition, as shown in FIGS. 8A to 9C, although the inner surfaces of the depressions 62b may have substantially rectangular tubular shapes that conform to the outer surfaces 41a, 42a, 43a, and 44a of the piezoelectric elements 41, 42, 43, and 44, as shown in FIGS. 10A to 10C, the inner surfaces of the depressions 62b may have shapes that abut against side surfaces of the piezoelectric elements 41, 42, 43, and 44.

In other words, engagement depressions 62d may be formed in the inner circumferential surfaces of the depressions 62b of the pressing portions 62, wherein the engagement depressions 62d have shapes that are complementary to those of at least one portion at the outer surfaces 41a, 42a, 43a, and 44a in the distal-end portions or proximal-end portions of the piezoelectric elements 41, 42, 43, and 44, and the at least one portion engages with the engagement depressions 62d.

By doing so, because the piezoelectric elements 41, 42, 43, and 44 and the pressing portions 62 are positioned with respect to each other also in the rotational direction about the longitudinal axis, it is possible to further enhance the assembly precision between the piezoelectric elements 41, 42, 43, and 44 and the pressing portions 62.

In this embodiment, although the vibration propagating portion 3 is formed of a rectangular tubular member, the vibration propagating portion 3 may be formed of a cylindrical member. In this case also, the piezoelectric elements 41 and 43 and the piezoelectric elements 42 and 44 are secured to the outer circumferential surface of the vibration propagating portion 3 with equal spacings therebetween in the circumferential direction so as to individually face each other in the X-direction and the Y-direction.

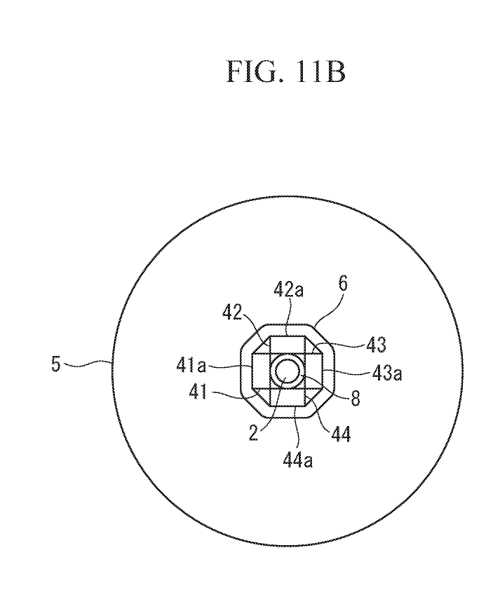

In this embodiment, although the piezoelectric elements 41, 42, 43, and 44 are secured to the outer circumferential surface of the optical fiber 2 via the vibration propagating portion 3, alternatively, as shown in FIGS. 11A and 11B, the piezoelectric elements 41, 42, 43, and 44 may directly be secured to the outer circumferential surface of the optical fiber 2.

In a modification in FIGS. 11A and 11B, a metal coating 8 is applied to at least a portion of the outer circumferential surface of the optical fiber 2 that is disposed at the interior of the vibration propagating portion 3. By doing so, it is possible to use solder or an epoxy-based adhesive to bond the optical fiber 2 and the piezoelectric elements 41, 42, 43, and 44. In addition, the securing portion 5 and the piezoelectric elements 41, 42, 43, and 44 may electrically be connected with each other via the metal coating 8 so that the securing portion 5 can serve as a common GND.

A first aspect of the present invention is an optical fiber scanner including: an optical fiber that guides light from a side of a proximal-end portion side to a side of a distal-end portion side along a longitudinal axis, the optical fiber emitting the light from the distal-end portion; a piezoelectric element that is secured to an outer circumferential surface of the optical fiber, the piezoelectric element, as a result of an alternating voltage being applied thereto, generating stretching vibrations in a direction along the longitudinal axis; and a pressing portion that, of an outer surface of the piezoelectric element positioned on the radially outer side of the optical fiber, presses down, radially inward, a portion corresponding to an antinode of the stretching vibrations in a direction along the longitudinal axis of the piezoelectric element.

With the present invention, when the piezoelectric element generates, as a result of the alternating voltage being applied thereto, the stretching vibrations in the longitudinal direction of the optical fiber, bending vibrations are excited in the optical fiber secured to the piezoelectric element, thus causing the distal end of the optical fiber to vibrate in a radial direction. By doing so, it is possible to scan the light emitted from the distal end of the optical fiber.

In this case, as a result of the piezoelectric element being pressed toward the optical fiber by the pressing portion, the piezoelectric element evenly comes into contact with the optical fiber. By doing so, it is possible to increase the vibration amplitude of the optical fiber by enhancing the efficiency at which the vibrations are propagated to the optical fiber from the piezoelectric element. Furthermore, by applying a pressing force to the piezoelectric element from the pressing portion, it is possible to supply a greater alternating voltage to the piezoelectric element, and thus, it is possible to further increase the vibration amplitude of the optical fiber. In particular, by providing the pressing portion at the position in the piezoelectric element corresponding to antinode of the stretching vibrations, because the vibrations at a maximum displacement position of the piezoelectric element are transmitted to the optical fiber, it is possible to more efficiently propagate the vibrations.

In the above-described first aspect, the pressing portion may press down only a distal-end portion and a proximal-end portion or only one of the distal-end portion and the proximal-end portion in a direction along the longitudinal axis of the outer surface of the piezoelectric element.

By providing the pressing portion at the distal-end portion and/or the proximal-end portion of the piezoelectric element corresponding to the antinode of the stretching vibrations, because the vibrations at the maximum displacement position of the piezoelectric element are transmitted to the optical fiber, it is possible to more efficiently propagate the vibrations.

In the above-described first aspect, the pressing portion may include an annular member that wraps around the periphery of the optical fiber and the piezoelectric element, and an abutting surface on which an end portion of the piezoelectric element abuts in the direction along the longitudinal axis may be formed in an inner surface of the pressing portion.

As a result of the end portion of the piezoelectric element abutting against the abutting surface, it is possible to enhance the assembly precision between the piezoelectric element and the pressing portion in the direction along the longitudinal axis, and thus, it is possible to more efficiently propagate the vibrations.

In the above-described first aspect, the inner surface of the pressing portion may have a shape that conforms to the outer surface of an end portion of the piezoelectric element.

In the above-described first aspect, an engagement depression may be formed in the inner surface of the pressing portion, wherein the engagement depression may have a complementary shape to that of at least one portion at the outer surface of the end portion of the piezoelectric element, and the at least one portion may engage with the engagement depression.

By doing so, as a result of at least a portion of the side surface of the piezoelectric element abutting against a surface of the engagement depression, because the pressing portion is positioned with respect to the piezoelectric element also in a rotating direction about the longitudinal axis of the optical fiber, it is possible to further enhance the assembly precision between the piezoelectric element and the pressing portion.

The above-described first aspect may be provided with a lead line that is connected to the outer surface of the piezoelectric element and that supplies the alternating voltage to the piezoelectric element, wherein the pressing portion may cover the outer surface so as to sandwich the lead line between the outer surface of the piezoelectric element and the pressing portion.

By doing so, it is possible to stably maintain the connection of the piezoelectric element to the lead line.

A second aspect of the present invention is an illumination apparatus including: an optical fiber scanner according to the above-described first aspect; and a light source portion that is connected to the proximal-end portion of the optical fiber, the light source portion supplying the light to the optical fiber.

A third aspect of the present invention is an observation apparatus including: an illumination apparatus according to the above-described second aspect; a light detecting portion that detects return light returning from an imaging subject, which is generated by the imaging subject being irradiated with the light coming from the illumination apparatus; and a voltage supplying portion that supplies the alternating voltage to the piezoelectric element.

The present invention affords an advantage in that it is possible to enhance the efficiency at which vibrations are propagated to an optical fiber from a piezoelectric element, thus increasing the vibration amplitude of the optical fiber.

### REFERENCE SIGNS LIST

1 optical fiber scanner
2 optical fiber
41, 42, 43, 44 piezoelectric element
41a, 42a, 43a, 44a outer surface
6, 61, 62 pressing portion
62c abutting surface
62d engagement depression
7A, 7B lead line
10 illumination apparatus
12 light source (light source portion)
20 light detecting portion
30 drive control apparatus (voltage supplying portion)
100 observation apparatus

1. An optical fiber scanner comprising:
an optical fiber that guides light from a side of a proximal-end portion to a side of a distal-end portion along a longitudinal axis, the optical fiber emitting the light from the distal-end portion;
a piezoelectric element that is secured to an outer circumferential surface of the optical fiber, the piezoelectric element, as a result of an alternating voltage being applied thereto, generating stretching vibrations in a direction along the longitudinal axis; and
a pressing portion that, of an outer surface of the piezoelectric element positioned on the radially outer side of the optical fiber, presses down, radially inward, a portion corresponding to an antinode of the stretching vibrations in a direction along the longitudinal axis of the piezoelectric element.

2. An optical fiber scanner according to claim 1, wherein the pressing portion presses down only a distal-end portion and a proximal-end portion in the direction along the longitudinal axis of the outer surface of the piezoelectric element.

3. An optical fiber scanner according to claim 1, wherein the pressing portion presses down only the proximal-end portion in the direction along the longitudinal axis of the outer surface of the piezoelectric element.

4. An optical fiber scanner according to claim 1, wherein the pressing portion presses down only the distal-end portion in the direction along the longitudinal axis of the outer surface of the piezoelectric element.

5. An optical fiber scanner according to claim 2,
wherein the pressing portion comprises an annular member that wraps around the periphery of the optical fiber and the piezoelectric element, and
an abutting surface on which an end portion of the piezoelectric element abuts in the direction along the longitudinal axis is formed in an inner surface of the pressing portion.

6. An optical fiber scanner according to claim 5, wherein the inner surface of the pressing portion has a shape that conforms to the outer surface of an end portion of the piezoelectric element.

7. An optical fiber scanner according to claim 6, wherein an engagement depression is formed in the inner surface of the pressing portion, wherein the engagement depression has a complementary shape to that of at least one portion at the outer surface of the end portion of the piezoelectric element, and the at least one portion engages with the engagement depression.

8. An optical fiber scanner according to claim 2, further comprising:
a lead line that is connected to the outer surface of the piezoelectric element and that supplies the alternating voltage to the piezoelectric element,
wherein the pressing portion covers the outer surface so as to sandwich the lead line between the outer surface of the piezoelectric element and the pressing portion.

9. An illumination apparatus comprising:
an optical fiber scanner according to claim 1; and
a light source that is connected to the proximal-end portion of the optical fiber, the light source portion supplying the light to the optical fiber.

10. An observation apparatus comprising:
an illumination apparatus according to claim 9;
a light detector that detects return light returning from an imaging subject, which is generated by the imaging subject being irradiated with the light coming from the illumination apparatus; and
a voltage supplying portion that supplies the alternating voltage to the piezoelectric element.

11. An optical fiber scanner according to claim 3,
wherein the pressing portion comprises an annular member that wraps around the periphery of the optical fiber and the piezoelectric element, and
an abutting surface on which an end portion of the piezoelectric element abuts in the direction along the longitudinal axis is formed in an inner surface of the pressing portion.

12. An optical fiber scanner according to claim 4,
wherein the pressing portion comprises an annular member that wraps around the periphery of the optical fiber and the piezoelectric element, and
an abutting surface on which an end portion of the piezoelectric element abuts in the direction along the longitudinal axis is formed in an inner surface of the pressing portion.

13. An optical fiber scanner according to claim 11, wherein the inner surface of the pressing portion has a shape that conforms to the outer surface of an end portion of the piezoelectric element.

14. An optical fiber scanner according to claim 12, wherein the inner surface of the pressing portion has a shape that conforms to the outer surface of an end portion of the piezoelectric element.

15. An optical fiber scanner according to claim 13, wherein an engagement depression is formed in the inner surface of the pressing portion, wherein the engagement depression has a complementary shape to that of at least one portion at the outer surface of the end portion of the piezoelectric element, and the at least one portion engages with the engagement depression.

16. An optical fiber scanner according to claim 14, wherein an engagement depression is formed in the inner surface of the pressing portion, wherein the engagement depression has a complementary shape to that of at least one portion at the outer surface of the end portion of the piezoelectric element, and the at least one portion engages with the engagement depression.

17. An optical fiber scanner according to claim 3, further comprising:
a lead line that is connected to the outer surface of the piezoelectric element and that supplies the alternating voltage to the piezoelectric element,
wherein the pressing portion covers the outer surface so as to sandwich the lead line between the outer surface of the piezoelectric element and the pressing portion.
